(19) United States
(12) Patent Application Publication
(10) Pub. No.: US 2026/0364306 A1
(43) Pub. Date: Nov. 19, 2026

(54) URINARY CATHETER WITH INTRODUCING AID

(71) Applicant: Hollister Incorporated, Libertyville, IL (US)

(72) Inventors: Satwinder S. Panesar, Foxford (IE); David J. Farrell, Ballina (IE)

(21) Appl. No.: 19/477,396

(22) PCT Filed: Apr. 22, 2024
(86) PCT No.: PCT/US2024/025675

§ 371 Date: Oct. 21, 2025

Related U.S. Application Data
(60) Provisional application No. 63/497,926, filed on Apr. 24, 2023.

(51) Int. Cl. A61M 25/00

(52) U.S. Cl. CPC A61M 25/0017; A61M 25/002; A61M 2025/0046; A61M 2205/0205; A61M 2205/051

(57) ABSTRACT

A catheter product including a urinary catheter with an introducing aid. More particularly, a urinary catheter with an antimicrobial agent associated with the introducing aid.

The present application claims the benefit and priority to U.S.

Provisional Application 63/497,926, filed on Apr. 24, 2023, which is hereby incorporated herein by reference.

### FIELD OF THE INVENTION

This disclosure generally relates to a urinary catheter product including an introducing aid. More particularly, this disclosure relates to an intermittent urinary catheter product with an antimicrobial agent associated with an introducing aid.

### BACKGROUND

Intermittent catheterization is a good option for many users who suffer from various abnormalities of the urinary system. With the advent of intermittent urinary catheters, individuals with problems associated with the urinary system can conveniently self-catheterize to drain the individual's bladder. Individuals who suffer from urinary incontinence will self-catheterize several times a day.

Although catheters are typically prepared in sterile environments and are provided with safe handling instructions, catheter users are at risk of contracting a urinary tract infection due to several different factors. For example, the catheters may become contaminated during use and introduce bacteria into the urethra. The catheter also may carry bacteria along the urinary tract.

Therefore, there is a need for methods and devices that reduce the risk of urinary tract infections.

### SUMMARY OF INVENTION

There are several aspects of the present subject matter which may be embodied separately or together in the subject matter claimed below. These aspects may be employed alone or in combination with other aspects of the subject matter described herein, and the description of these aspects together is not intended to preclude the use of these aspects separately or the claiming of such aspects separately or in different combination as set forth in the claims appended hereto.

In one aspect a urinary catheter product is provided. The urinary catheter product includes an intermittent urinary catheter. The intermittent urinary catheter includes a catheter tube with a proximal insertion end and a distal drainage end and a catheter tube outer surface and a catheter tube inner surface, wherein the catheter tube inner surface defines a drainage lumen. The intermittent urinary catheter also includes a hydrophilic coating on the catheter tube outer surface. The intermittent urinary catheter also includes an introducing aid, wherein the introducing aid assists with introducing the catheter tube into a urethra. The introducing aid includes an introducing aid inner surface defining an introducing aid lumen configured to receive the catheter tube therethrough. Additionally, the intermittent urinary catheter includes a layer of antimicrobial agent located on the introducing aid inner surface, wherein the antimicrobial agent is deposited on the hydrophilic coating as the catheter tube moves through the introducing aid lumen.

In another aspect, a method for making an antimicrobial catheter is provided. The method includes dissolving a polysaccharide into an aqueous solution, adding an antimicrobial agent to the aqueous solution, drying the aqueous solution to create an antimicrobial gel, applying a layer of the antimicrobial gel to an inner surface of an introducing aid associated with the catheter, and releasing the antimicrobial agent from the antimicrobial gel.

### BRIEF DESCRIPTION OF DRAWINGS

FIG. 1 is a perspective view of an embodiment of a catheter product.

FIG. 2 is a perspective view of an embodiment of an introducing aid.

FIG. 3 is a cross-sectional view of a portion of the introducing aid of FIG. 3 with a catheter tube within the introducing aid.

FIG. 4 is a perspective view of another embodiment of a catheter product.

FIG. 5 is a cross-sectional view of a gripper with a catheter tube.

### DETAILED DESCRIPTION

A more detailed description of the device in accordance with the present disclosure is set forth below. It should be understood that the description of the specific devices below is intended to be exemplary, and not exhaustive of all possible variations or applications. Thus, the scope of the disclosure is not intended to be limiting and should be understood to encompass variations or embodiments that would occur to persons of ordinary skill.

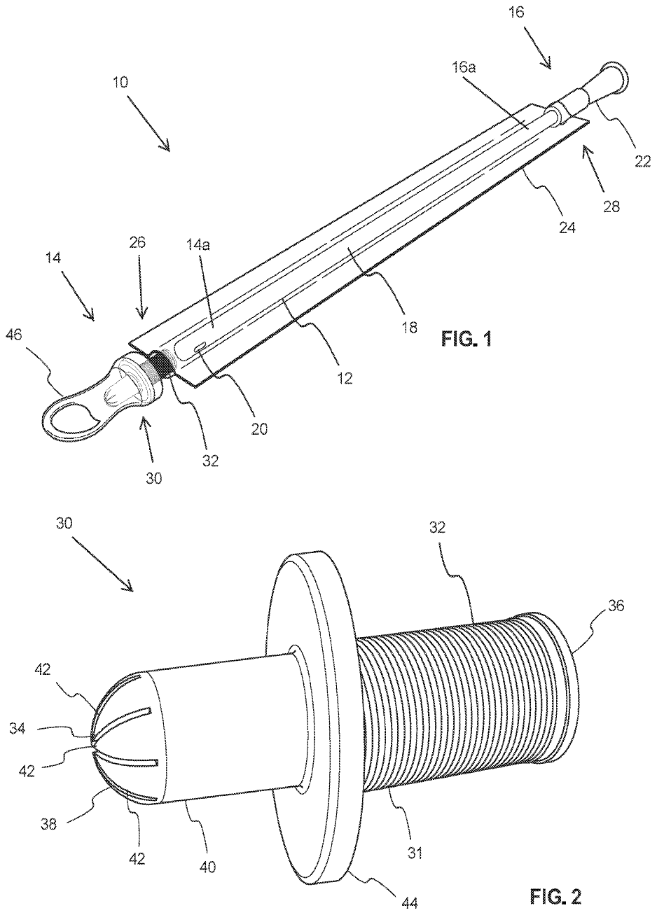

FIG. 1 shows an embodiment of a urinary catheter product 10. Optionally, the urinary catheter product 10 may be a sleeved intermittent urinary catheter product. The urinary catheter product 10 includes a catheter tube 12 having a catheter proximal insertion end 14 and a catheter distal drainage end 16. The catheter tube 12 also includes a tube outer surface 18 and a tube inner surface. The tube inner surface defines a lumen extending from the catheter tube proximal insertion end 14a to the catheter tube distal drainage end 16a.

The urinary catheter tube 12 may include a hydrophilic polymer coating on the tube outer surface 18. The hydrophilic polymer coating may be configured to absorb a hydration medium to lubricate the catheter for easing insertion into a urethra.

Additionally, the catheter tube proximal insertion end 14a includes one or more openings 20 for receiving urine in communication with the lumen. In one embodiment, the one or more openings 20 for receiving urine may be an eyelet. In another embodiment, the one or more openings 20 for receiving urine may be multiple eyelets. The one or more openings 20 for receiving urine may vary in shape, size, and location without departing from the scope of the disclosure.

Furthermore, the catheter tube distal drainage end 16a includes a drainage opening for draining urine from the lumen. In one embodiment, the catheter distal drainage end 16 includes a drainage member 22. The drainage member 22 may be attached to the catheter tube distal drainage end 16a and may be in fluid communication with the drainage opening. The drainage member 22 may be, for example, but not limited to, a funnel or connector for connection to a drainage bag (not shown). Other drainage members known in the art may be used without departing from the scope of the disclosure.

The urinary catheter product 10, optionally, may include a sleeve 24. The sleeve 24 may be a protective or barrier sleeve. The sleeve 24 has a sleeve proximal end 26 and a sleeve distal end 28. The sleeve 24 may define an internal cavity in which the catheter tube 12 may be located. In an embodiment, the sleeve 24 surrounds at least a portion of the catheter tube 12. In another embodiment, the sleeve 24 extends over and surrounds the length of the catheter tube 12.

In an embodiment, an introducing aid 30 may be located at the sleeve proximal end 26. When an introducing aid 30 is present, the sleeve proximal end 26 may be attached to a barrel portion 32 of the introducing aid 30 by, for example, welding or adhesive. The sleeve distal end 28 may be attached to the drainage member 22 or the catheter tube 12. The sleeve distal end 28 may be attached to the drainage member 22 or catheter tube 12 by, for example, welding or adhesive.

The sleeve 24 may be made of a flexible material which may be vapor permeable or vapor impermeable, depending on the desired use and packaging. The material of the sleeve 24 also may be liquid impermeable or liquid permeable. The sleeve 24 may be formed of any of a variety of thin, flexible polymeric film materials, such as polyethylene, plasticized PVC, or polypropylene, but elastomeric film materials such as polyurethane, and particularly elastomeric hydrogel materials, may be suitable. In some embodiments, the sleeve 24 may contain a hydration medium.

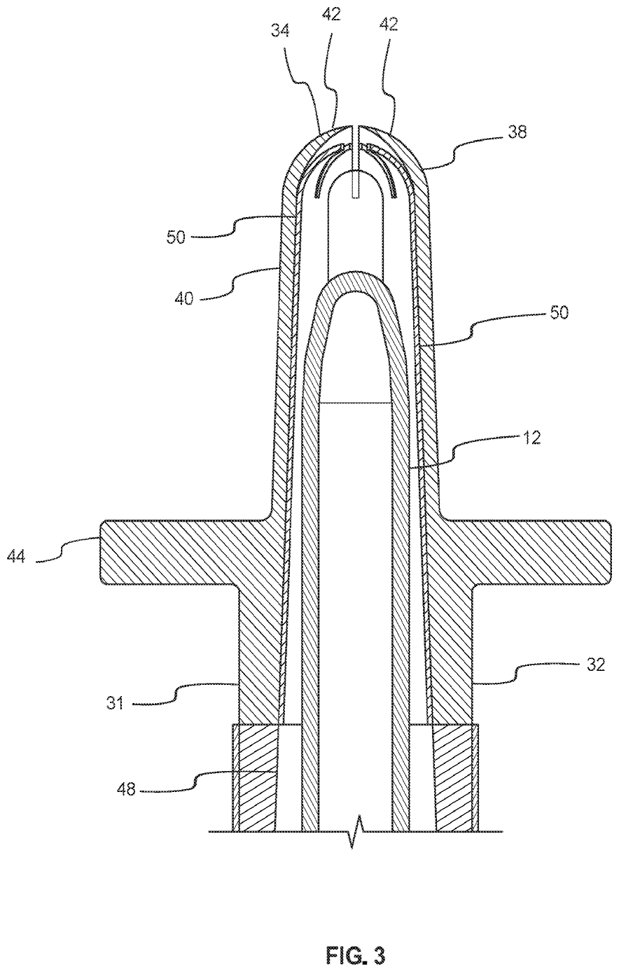

Turning to FIG. 2, an embodiment of an introducing aid 30 is shown. The introducing aid 30 includes a body 31 with an introducing aid proximal end 34 and an introducing aid distal end 36. An introducing aid inner surface 48 (shown in FIG. 3) defines a lumen extending from the introducing aid proximal end 34 to the introducing aid distal end 36. The introducing aid proximal end 34 includes an introducer tip 38. The introducing aid proximal end 34 may also include a portion 40 extending distally from the introducer tip 38.

The introducer tip 38 has a proximal end configured to be openable. For example, the introducer tip 38 may be opened by advancing the catheter tube 12 therethrough. In an embodiment, the proximal end of the introducer tip 38 is defined by one or more slits between one or more flexible petals 42. The petals 42 may move, bend, and/or resiliently deform from the generally closed configuration shown in FIG. 2 to an open configuration when a catheter tube is advanced therethrough. In another embodiment, the introducer tip 38 may include a rupturable proximal end.

Additionally, the introducing aid distal end 36 includes a barrel portion 32 that has an opening for receiving the catheter tube 12. The barrel portion 32 of the introducing aid 30 may include a textured outer surface to assist a user in gripping the introducing aid 30.

In some embodiments, the introducing aid 30 may include an intermediate flange 44. The flange 44 may contact the user around the urethral opening and act as a stop to limit the insertion of the introducer tip 38 into the urethra. The flange 44 also may aid in placement of the user's fingers on the barrel portion 32.

In some embodiments, the catheter product 10 may include a cap 46 (shown in FIG. 1). The cap 46 includes a cavity configured to receive at least the introducer tip 38 of the introducing aid 30. The cap 46 may be attached to the introducing aid 30 via, for example, but not limited to, a friction fit. For example, the cap 46 may be attached to the intermediate flange 44.

Furthermore, the urinary catheter product 10 may include an antimicrobial agent associated with the introducing aid 30. As shown in FIG. 3, a layer of antimicrobial agent 50 may be located on an inner surface 48 of the introducing aid 30. In an embodiment, the antimicrobial agent layer 50 may be located on the inner surface of the introducer tip 38. For example, the antimicrobial agent layer 50 may be located on the inner surface of the petals 42. Optionally or in addition to the antimicrobial agent on the petals 42, the antimicrobial agent layer 50 may be located on an inner surface of the portion 40 extending distally from the introducer tip 38. For example, in some alternatives, the antimicrobial agent layer 50 may be located on the entire inner surface of the introducing aid 30 proximal end, including the inner surface of the introducer tip 38 and of the portion 40 extending distally therefrom, or the layer 50 may be located on selected portions thereof. In other alternatives, the antimicrobial agent layer 50 may be located on the entire inner surface 48 of the introducing aid 30, including barrel portion 32, introducer tip 38 and the portion 40 extending distally therefrom. In some alternatives, antimicrobial agent layer 50 may be continuous, and in other alternatives, layer 50 may include a plurality of spaced apart segments along inner surface 48 having breaks therebetween.

The antimicrobial agent layer 50 may be applied to the introducing aid 30 prior to assembly of the catheter product 10. For example, the antimicrobial agent layer 50 may be applied to the inner surface 48 of the introducing aid 30 by spray coating, painting, or other methods known in the art.

The antimicrobial agent may be a compound that at least reduces the risk of a urinary tract infection. It may be a biocide, an agent that reduces or prevents adhesion to a surface, an agent that reduces or prevents microbial proliferation, etc. In one embodiment, the antimicrobial agent is a biocide. Suitable biocides may be silver, antibiotics with biocidal properties such as vancomycin, sparfloxacin, minocycline, and rifampin among others, and poly(catechins)-antibiotic conjugates such as polycatechin with trimethoprim (TMP) and/or polycatechin with TMP and sulfamethoxazole (SMZ). In another embodiment, the anti-microbial agent may have steric repulsion, electrostatic repulsion, and/or low surface energy properties. In yet another embodiment, the antimicrobial agent may be an antifouling material. Suitable antifouling materials include, but are not limited to, hydrophilic materials such as SAM-OEG, PEG, and POEGMA; polyzwitterions such as polyMPC, polyCBMA, and polySBMA; phosphorylcholines; sulfobetaines; and carboxybetaines. In yet another embodiment, the antimicrobial agent may be any other suitable antimicrobial agent, such as chlorhexidine, amphotericin, bacteriophages, nitrofural, triclosan, antimicrobial peptides, and/or allantoin. In another embodiment, the antimicrobial agent may be an enzyme. Suitable enzymes include hydrolytic enzymes such as proteolytic enzymes (e.g., subtilisin and lysostaphin), polysaccharide hydrolyzing enzymes (e.g., alpha amylase, dispersin B, chitinases, beta-glucanases, lysozyme, and alginate lyase), DNases, and bacteriophage lysins. In yet another embodiment, the antimicrobial agent may be nitric oxide. In yet another embodiment, the antimicrobial agent may be liposomes such as spherical lipid vesicles that consist of one or more phospholipid bilayers. Other antimicrobial agents known in the art may be used without departing from the scope of the disclosure. In another embodiment, the antimicrobial agent may be a combination of any of the agents described herein, amongst others.

In an embodiment, the antimicrobial agent layer 50 may be applied to the introducing aid 30 as a coating, lubricant, grease, capsule, and/or sachet. For example, the antimicrobial agent may be incorporated in polyols (such as glycerol, PEG, etc.), mineral oils, silicone oils, vegetable oils, pastes, viscous liquids or semi-solids, jellies, gels, and/or hydrogels.

In another embodiment, the antimicrobial agent layer 50 may be an antimicrobial agent incorporated in a plurality of capsules (such as microcapsules) or in a gel, which may be a hard or soft gel. The gel or capsules may be applied to the introducing aid 30 prior to assembly of the catheter product 10. The gel or capsules may break down into a liquid when exposed to radiation during sterilization of the catheter product, thus releasing the antimicrobial agent and providing a liquid film on the inner surface of the introducing aid 30. For example, the gel or capsules may breakdown when exposed to electron beam or Gamma radiation used for sterilization. By liquefying the gel or capsules, the antimicrobial agent may be released and allowed to smear onto the hydrated hydrophilic coating of the catheter tube 12 as it is pushed through the introducing aid 30. This way, the antimicrobial agent may be directly applied to the urethra surface as the catheter tube 12 is inserted via the introducer tip 38. In another alternative, the gel or capsules may break down and release the antimicrobial agent due to exposure to a temperature, a pH, biodegradation with time, and/or heat.

In an embodiment when the antimicrobial agent layer 50 includes a gel coating, the gel coating may include gellan gum and citric acid.

In an embodiment when the antimicrobial agent layer 50 includes microcapsules, the microcapsules may include an encapsulate encapsulating the antimicrobial agent. The encapsulate may be made of a variety of materials, such as starch, starch derivatives, proteins, gelatins, gums, lipids, natural and synthetic polymers, or any combination of such encapsulates. Examples of natural polymers include, but are not limited to, protein-based polymers and polysaccharide-based polymers. The natural based polymers may be highly biodegradable. Suitable protein-based polymers include, but are not limited to, albumin and gelatin. Suitable polysaccharide-based polymers include, but are not limited to, agarose, alginate, hyaluronic acid, dextran, chitosan, and gellan gum, among others. Examples of synthetic polymers include, but are not limited to, poly(ε-caprolactone), poly(lactic-co-glycolic acid), and polyethylene glycol. Other encapsulates known in the art may be used without departing from the scope of the disclosure.

The microcapsules may be made by dissolving the polysaccharide in a solute to form a homogenous solution. In one alternative, gellan gum at greater than 1.0% w/w is dissolved in deionized water at a temperature that is greater than 75 °C. Once dissolved, a selected amount/concentration of the antimicrobial agent may be added to the polysaccharide aqueous solution. After the antimicrobial agent has been added, the solution is dried to encapsulate the antimicrobial agent in the polysaccharide. For example, the solution may be cooled to a selected temperature to solidify the polysaccharide and to form a microcapsule encapsulating the anti-microbial therein. When gellan gum is the polysaccharide, the solution may be cooled to below 37 °C, thereby commencing solidification of the gellan gum. In an alternative, to assist with drying the polysaccharide encapsulate at temperatures above 37 °C, spray-drying and spin-drying methods may be employed. Other methods of encapsulation may also be used, such as freeze drying, vacuum drying, extrusion, spray cooling, fluidized bed coating, centrifugal extrusion, co-crystallization, molecular inclusion, pan coating, air suspension coating, coacervation, solvent evaporation, and polymerization among other methods known in the art. Furthermore, in some embodiments, about 0.05% w/w to 0.25% w/w citric acid may be added to assist in hardening the gel or capsule to solid form.

In another embodiment, the microcapsules may be those described in U.S. Provisional Application No. 63/339172 filed on May 6, 2022, and in International Application PCT/US2023/066163, filed on Apr. 25, 2023, the entireties of which are incorporated herein.

To use the catheter product 10, a user may grasp the barrel portion 32 of the introducing aid 30 and insert the introducer tip 38 into the urethra. The user then may grasp the catheter tube 12 through the sleeve 24 and advance the catheter tube 12 through the introducing aid 30 and into and through the urethra until the one or more openings 20 enter the bladder. As the catheter tube 12 advances through the introducing aid 30, the antimicrobial agent layer 50 on the inner surface 48 of the introducing aid 30 may be deposited onto the hydrophilic coating of the catheter tube 12.

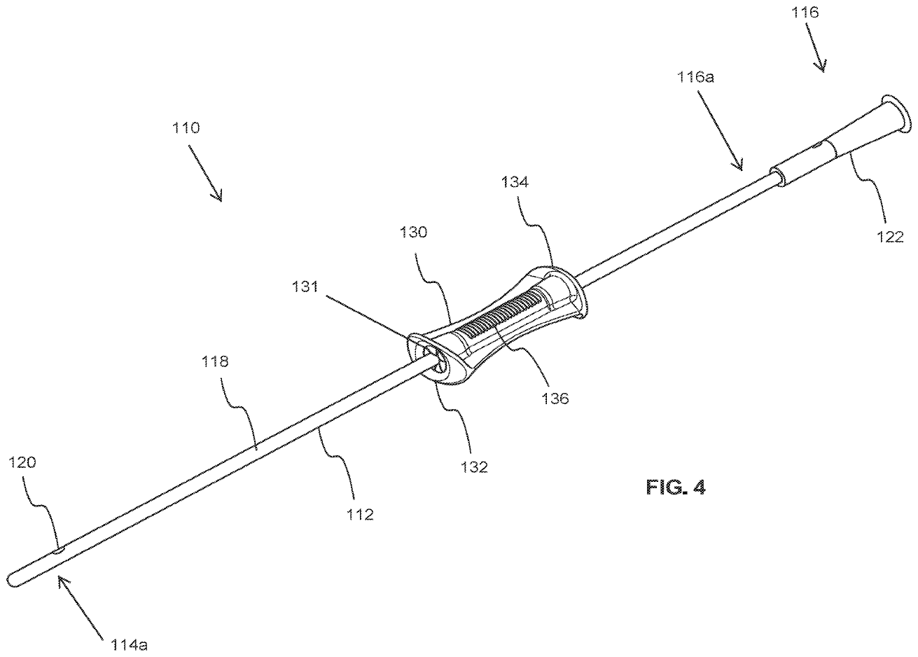

Turning to FIG. 4, another embodiment of a urinary catheter product 110 is shown. Catheter product 110 may include similar features to urinary catheter product 10 described herein. The urinary catheter product 110 may include a catheter tube 112 having a catheter tube proximal insertion end 114a and a catheter tube distal drainage end 116a. The catheter tube 112 includes a tube outer surface 118 and a tube inner surface. The tube inner surface defines a lumen extending from the catheter tube proximal insertion end 114a to the catheter tube distal drainage end 116a. The catheter tube proximal insertion end 114a includes one or more openings 120 for receiving urine in communication with the lumen. In one embodiment, the one or more openings 120 for receiving urine may be an eyelet. In another embodiment, the one or more openings 120 for receiving urine may be multiple eyelets. The one or more openings 120 for receiving urine may vary in shape, size, and location without departing from the scope of the disclosure.

Furthermore, the catheter tube distal drainage end 116a includes a drainage opening for draining urine from the lumen. In one embodiment, the distal drainage end 116 of the catheter product 110 includes a drainage member 122.

The drainage member 122 may be attached to the catheter tube distal drainage end 116a and may be in fluid communication with the drainage opening. The drainage member 122 may be, for example, but not limited to, a funnel or connector for connection to a drainage bag (not shown). Other drainage members known in the art may be used without departing from the scope of the disclosures.

The urinary catheter product 110 may include a gripper 130. A gripper 130 may be provided to assist the user with handling the catheter product 110. For example, a user may grip the gripper 130 to handle and to aid in directing the insertion of the catheter tube 112 into the urethra.

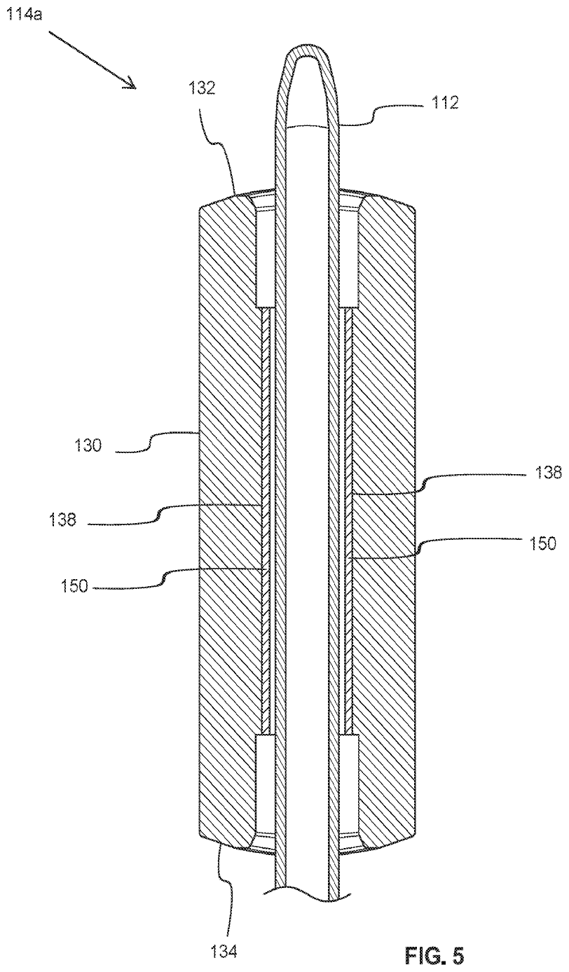

Gripper 130 includes a gripper proximal end 132 and a gripper distal end 134. The gripper 130 also includes a gripper outer surface 136 and a gripper inner surface 138 (shown in FIG. 5). The gripper inner surface 138 defines a lumen extending from the gripper proximal end 132 to the gripper distal end 134. The catheter tube 112 may be positioned within the lumen of the gripper 130. The gripper outer surface 136 may be textured to aid the user with gripping or as an indication of gripping location. The gripper 130 may be moveable along the length of the catheter tube 112. Furthermore, the gripper 130 may be flexible or elastic such that the user may squeeze the gripper 130 to grasp the catheter tube 112. When the user releases the gripper 130, the gripper 130 substantially returns to its original shape to allow movement or sliding of the gripper 130 along the catheter tube 112. Optionally, the gripper may include flaps or petals 131 that contact catheter tube 112 to assist in maintaining the gripper 130 in place along the catheter tube 112.

In an embodiment, the gripper 130 may include an antimicrobial agent. The antimicrobial agent may be a layer of antimicrobial agent 150 located anywhere along the gripper inner surface 138. The antimicrobial agent layer 150 may cover the entire inner surface 138 or selected portions thereof. The antimicrobial agent layer 150 may be continuous or include a plurality of spaced apart segments having breaks therebetween. The antimicrobial agent may be any of the antimicrobial agents described herein. For example, the antimicrobial agent layer 150 may be an antimicrobial agent incorporated in a plurality of capsules (such as microcapsules) or in a gel, which may be a hard or soft gel. The gel or capsules may break down and release the antimicrobial agent when exposed to irradiation, for instance, electron beam or Gamma radiation used to sterilize the catheter product 110.

The antimicrobial agent layer 150 may be applied to the gripper 130 prior to assembly of the catheter product 110. For example, the antimicrobial agent layer 150 may be applied to the gripper inner surface 138 by spray coating, painting, or other methods known in the art.

To use the catheter product 110, a user may move the gripper 130 towards the catheter tube proximal end 114 and squeeze the gripper 130 to grasp the catheter tube 112. While grasping the catheter tube 112, the user may then insert the catheter tube 112 into the urethra. After insertion, the user may then move the gripper 130 in the distal direction, squeeze the gripper 130, and advance the catheter tube 112 further into the urethra. This may be repeated until the catheter tube 112 enters the bladder. As the gripper 130 moves along the length of the catheter tube 112, the antimicrobial agent on the gripper inner surface 138 may be deposited onto the hydrophilic coating of the catheter tube 112.

It will be understood that the embodiments and examples described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1. A urinary catheter product comprising:
an intermittent urinary catheter including:
a catheter tube with a proximal insertion end and a distal drainage end, and a catheter tube outer surface and a catheter tube inner surface, wherein the catheter tube inner surface defines a drainage lumen;
a hydrophilic coating on the catheter tube outer surface;
an introducing aid, wherein the introducing aid assists with introducing the catheter tube into a urethra, the introducing aid comprising an introducing aid inner surface defining an introducing aid lumen configured to receive the catheter tube therethrough; and
a layer of antimicrobial agent located on the introducing aid inner surface, wherein the antimicrobial agent is deposited on the hydrophilic coating as the catheter tube moves through the introducing aid lumen.

2. The urinary catheter product of claim 1, wherein the layer comprises a coating and the antimicrobial agent is incorporated into the coating.

3. The urinary catheter product of claim 1, wherein the layer comprises microcapsules and the antimicrobial agent is incorporated into the microcapsules.

4. The urinary catheter product of claim 1, wherein the layer comprises a polysaccharide.

5. The urinary catheter product of claim 4, wherein the polysaccharide is gellan gum.

6. The urinary catheter product of claim 1, wherein the layer further comprises citric acid.

7. (canceled)

8. The urinary catheter product of claim 1, wherein the antimicrobial agent comprises a biocide, an antifouling material, an anti-microbial, an enzyme, a nitric oxide agent, a liposome, or any combination of such agents.

9. The urinary catheter product of claim 3, wherein the microcapsules comprise an encapsulate, wherein the encapsulate comprises starch, starch derivatives, proteins, gelatins, gums, lipids, natural polymers, synthetic polymers, or any combination of such encapsulates.

10. The urinary catheter product of claim 1, wherein the antimicrobial agent is released upon the layer being exposed to electron beam irradiation.

11. The urinary catheter product of claim 1, wherein a body of the introducing aid comprises a proximal introducer tip configured to be inserted into the urethral opening.

12. The urinary catheter product of claim 11, wherein the layer of antimicrobial agent is located on an inner surface of the proximal introducer tip.

13. (canceled)

14. (canceled)

15. A method for making an antimicrobial catheter comprising:
dissolving a polysaccharide into an aqueous solution;
adding an antimicrobial agent to the aqueous solution;
drying the aqueous solution to create an antimicrobial gel;
applying a layer of the antimicrobial gel to an inner surface of an introducing aid associated with the catheter; and
releasing the antimicrobial agent from the antimicrobial gel.

16. The method of claim 15, wherein the catheter comprises
a catheter tube with a proximal insertion end and a distal drainage end, a catheter tube outer surface and a catheter tube inner surface;
a hydrophilic coating on the catheter tube outer surface; and
the introducing aid, wherein the introducing aid comprises an introducing aid inner surface defining an introducing aid lumen configured to receive the catheter tube therethrough.

17. The method of claim 16, further comprising depositing the antimicrobial agent on the hydrophilic coating as the catheter tube moves through the introducing aid lumen.

18. The method of claim 15 wherein, the layer comprises a coating.

19. The method of claim 15, wherein the layer comprises microcapsules.

20. The method of claim 15 , wherein the polysaccharide is gellan gum.

21. The method of claim 15, wherein the gel further comprises citric acid.

22. (canceled)

23. The method of claim 15, wherein the antimicrobial agent comprises a biocide, an antifouling material, an anti-microbial, an enzyme, a nitric oxide agent, a liposome, or any combination of such agents.

24. The method of claim 15, wherein the antimicrobial agent is released upon the gel being exposed to electron beam irradiation.

25. (canceled)

26. (canceled)

27. (canceled)

28. (canceled)
